(19) United States
(12) Patent Application Publication
(10) Pub. No.: US 2009/0216169 A1
(43) Pub. Date: Aug. 27, 2009

(54) Layered product with adhesive dressing for non-touch application

(76) Inventors: Grazyna Hansen, Frederiksberg (DK); Jan Marcussen, Taastrup (DK); Bjoern Christiansen, Hilleroed (DK); Anders Christian Nielsen, Copenhagen (DK)

Correspondence Address: JACOBSON HOLMAN PLLC, 400 SEVENTH STREET N.W., SUITE 600, WASHINGTON, DC, 20004, US

(21) Appl. No.: 11/991,163

(22) PCT Filed: Aug. 31, 2006
(86) PCT No.: PCT/DK2006/000471

§ 371 Date: Feb. 24, 2009

(30) Foreign Application Priority Data
Aug. 31, 2005 (DK) PA 2005 012078

(51) Int. Cl. A61F 13/02; A61B 19/02; B67B 7/00

(52) U.S. Cl. 602/48; 602/57; 206/440; 53/492

(57) ABSTRACT

A layered product comprising an adhesive dressing allowing transfer of one or more elements, e.g. elements containing an active substance or a hydrogel, to the adhesive surface of the adhesive dressing immediately before the adhesive dressing is applied to the surface, e.g. the skin, of a living being.

The present invention relates to a layered product comprising an adhesive dressing suitable for non-touch application, as well as a package containing said layered product.

The construction of the layered product of the invention allow transfer of one or more elements, e.g. elements containing an active substance or a hydrogel, to the adhesive surface of the adhesive dressing immediately before or during the adhesive dressing is applied to the surface of a living being, e.g. the skin, of a living being.

### Background of the Invention

Adhesive dressings for non-touch application are known. The known adhesive dressings are constructed in various ways, typically having one or more release layers with folded structures or extending tab members, which allow non-touch exposure of the adhesive layer and non-touch application of the adhesive dressing to the skin.

It is also known to use adhesive dressings for topical administration of active substances.

U.S. Pat. No. 5,059,189 describes a method for the preparation of an adhesive dressing carrying a pharmaceutically active ingredient. According to this reference, an adhesive dressing carrying a pharmaceutically active ingredient on its adhesive surface, may be constructed by preparing an adhesive dressing releasable attached to a release layer, dispersing a pharmaceutically active ingredient in a medium and coating or casting said dispersion on a second release layer, removing the first release layer from the adhesive dressing and laminating the exposed adhesive surface onto the second release layer, so than the pharmaceutically active ingredient contacts the exposed adhesive surface.

An advantage of the present invention compared to the adhesive dressing described above is that elements, such as the active substances, may be added to the adhesive surface Immediately before the adhesive dressing is applied to the skin or during application to the skin. This Is an advantage because migration of the active substance into the adhesive layer during storage may be eliminated or diminished.

Application of the active substance to the adhesive layer immediately before application provides a better control of the amount of active substance actually present on the adhesive surface, and the amount of substance may be reduced because migration into the adhesive layer is eliminated or diminished. The invention also has certain advantages in cases where an element, such as an active substance or a hydrogel, is not sufficiently compatible with the adhesive layer. Furthermore the layered product of the invention allow easy and accurately positioning of an element on the surface of the adhesive layer.

Furthermore, the present invention provides a package containing an adhesive dressing in a fixed position, which allows easy removal of the adhesive dressing from the package, and simultaneous application of e.g. an active substance to the adhesive layer. The integrated package may if desired be a sterile sealed package containing the adhesive dressing.

### SUMMARY OF THE INVENTION

Thus, the present invention relates to a layered product comprising:

(I) an adhesive dressing comprising an adhesive layer having a cover layer inseparably attached to one surface of said adhesive layer, the other surface of said adhesive layer consisting of a first adhesive zone, a second adhesive zone positioned distally to the first adhesive zone, and an adhesive mid zone positioned between the first and second adhesive zones,

(II) a first release layer releasable attached to the first adhesive zone of the adhesive dressing, said first release layer being folded upon itself to form a tap member and having the folded edge positioned at the boundary between the adhesive mid zone of and the first adhesive zone of the adhesive dressing,

(III) a second release layer having a surface area corresponding at least the surface area of the adhesive mid zone and optionally the first and/or second zones of the adhesive dressing, said second release layer optionally carrying one or more element(s) releasable attached to the second release layer on a first surface of the second release layer and wherein

a roller foil or third release layer having a tab member is releasable attached to at least the adhesive mid zone of the adhesive dressing, and in that the adhesive dressing and the second release layer are connected to each other by means allowing positioning of said first surface of the second release layer on the adhesive mid zone and optionally the second adhesive zone of the adhesive dressing by folding.

In one particular embodiment, the invention relates to a layered product as above wherein the second adhesive zone of the adhesive layer is releasable attached to an end portion of said second release layer.

According to this embodiment of the invention, the third release layer or roller foil is suitably a roller foil having two or more layers folded on top of each other and interposed between the second release layer and at least the adhesive mid zone of the adhesive dressing, where the outer surface of the first layer of said folded roller foil is releasable attached to the mid zone of the adhesive dressing and the folded edge between said first layer of the roller foil and the second layer of the roller foil is positioned at the boundary between the second adhesive zone and the adhesive mid zone of the adhesive layer.

The invention also relates to packages containing the above-mentioned layered products.

### BRIEF DESCRIPTION OF THE DRAWINGS

FIGS. 1-8 illustrate various embodiments of the invention.

### DETAILED DESCRIPTION OF THE INVENTION

In the following the meaning of certain words and phrases as used herein are defined.

In the present context, the term adhesive dressing should be interpreted in a broad sense, i.e. to include not only thin film dressings, but also any other kind of dressings, including other types of film dressings, dressing with absorbents (such as hydrocolloid adhesive dressings), blister healing dressings, callus relief dressings, bunion relief dressings, dressings for cuts or grazes, surgical drapes etc.

As used herein, the terms “ “attached to” means “releasable attached to” or “inseparably attached to”.

As used herein “releasable attached to”, means that the two layers may be separated without permanently deforming or damaging the layers involved.

As used herein, the expression “inseparably attached to” means that the layers cannot be separated while keeping both layers intact.

As used herein the word “sealed” means that the layers are attached to each other and forms a seal isolating the adhesive article from the environment.

A “tab member” means an extending portion or a folded portion of a layer, which is not attached to the adhesive layer of the adhesive dressing.

A “tab portion” means a folded portion and/or an end portion of the roller foil or bottom release layer, which extend beyond the area where the one or more elements is interposed between the roller foil(s) and the bottom release layer in a position, which is accessible and may be used to remove the roller foil or bottom release layer from said one or more element(s).

As used herein the phrase “providing a folding line,” means that it is possible to fold the layered product of the invention to place the second release layer on the adhesive layer of the adhesive dressing. The position of the folding line may be marked e.g. by a pre-folded line, a weakening line, a line drawn on the product, or by the line where layers are attached to each other. However, the position of the folding line need not be indicated on the product.

The phrase “providing a folding centre” has the same meaning as the phrase “providing a folding line” except that a folding line is a folding centre.

As used herein “roller foil” means a release layer, which may be rolled off the adhesive layer by pulling with an angle close to zero between the part of the roller which is rolled of and the part which is still resent on the adhesive layer, i.e. a release layer which is relatively flexible.

According, to one embodiment, the adhesive dressing and the second release layer lies in extension of each other and are connected to each other by one of their edges, the connection between the edges providing a folding line or folding centre allowing positioning of said first surface of the second release layer on the adhesive mid zone and optionally the second adhesive zone of the adhesive dressing.

According to another embodiment of the invention, the adhesive dressing and the second release layer lies in extension of each other and is carried on a support layer fixing the adhesive dressing and the second release layer at a distance from each other, where said support layer provides a folding line or center allowing positioning of said first surface of the second release layer on the adhesive mid zone and optionally the second adhesive zone of the adhesive dressing. When the support layer of the above-mentioned layered product is folded, the above-mentioned support layer may serve as a wrap for the layered product of the invention.

According the these embodiments of the invention, where the adhesive dressing and the second release layer lies in extension of each other, the roller foil or third release layer may be a single layer releasable attached to at least the entire adhesive mid zone of said adhesive dressing and optionally to any adhesive element(s) carried by the second release layer.

When the second release layer carries adhesive element(s), a roller foil or release layer release layer is suitably also releasable attached to said adhesive element (s).

The roller foil or third release layer may be a single layer covering only the adhesive layer of the adhesive dressing. A fourth release layer or foil may be releasable attached to adhesive element(s) carried by the first release layer.

The use of single layers, are preferred where the adhesive dressing and the second release layer lies in extension of each other.

The roller foil or the third release layer suitably has a tap member which does not adhere the adhesive portion of the adhesive dressing or to said adhesive element (s) carried by the first release layer.

Suitably, the tap member is an end portion of the roller foil or third release layer.

The tap portion may also be formed by a portion of the roller foil or third release layer folded three times, the first and third folding line being situated on top of and aligned with the folding line allowing positioning of the second release liner of the second release layer on the adhesive surface of the adhesive dressing by folding.

In a preferred one embodiment of the invention, the second adhesive zone of the adhesive layer is releasable attached to an end portion of said second release layer.

According to this embodiments of the invention, the roller foil or third release layer may or may not be interposed between the second release layer and the adhesive surface of the adhesive dressing, depending on whether or not the layered product is folded. Single layers of the third release layer/roller foil may become double layers when the layered product is folded and the third release layer or roller foil becomes interposed between the adhesive layer and the second release layer.

Preferably, the roller foil or third release layer is interposed between the second release layer and at least the adhesive mid zone of the adhesive dressing.

According to this embodiment of the invention, the roller foil or third release layer may be a single layer interposed between the second release layer and at least the adhesive mid zone of the adhesive dressing, or the roller foil or third release layer has two or more layers which are Interposed between the second release layer and at least the adhesive mid zone of the adhesive dressing.

According to a particularly preferred embodiment, the invention relates to a layered product comprising

(I) an adhesive dressing comprising an adhesive layer having a cover layer inseparably attached to one surface of said adhesive layer, the other surface of said adhesive layer consisting of a first adhesive zone, a second adhesive zone positioned distally to the first adhesive zone, and an adhesive mid zone positioned between the first and second adhesive zones,

(II) a first release layer releasable attached to the first adhesive zone of the adhesive dressing, said first release layer being folded upon itself to form a tap member and having the folded edge positioned at the boundary between the adhesive mid zone of and the first adhesive zone of the adhesive dressing,

(III) a second release layer having a surface area corresponding at least the surface area of the adhesive mid zone and optionally the first and/or second zones of the adhesive dressing, wherein a first surface of the second adhesive zone of the adhesive layer is releasable attached to an end portion of said second release layer and wherein said first surface of the second release layer optionally carries one or more element(s) releasable attached to the second release layer,

(IV) a roller foil having two or more layers folded on top of each other is interposed between the second release layer and at least the adhesive mid zone of the adhesive dressing, where the outer surface of the first layer of said folded roller foil is releasable attached to the mid zone of the adhesive dressing and the folded edge between said first layer of the roller foil and the second layer of the roller foil is positioned at the boundary between the second adhesive zone and the adhesive mid zone of the adhesive layer.

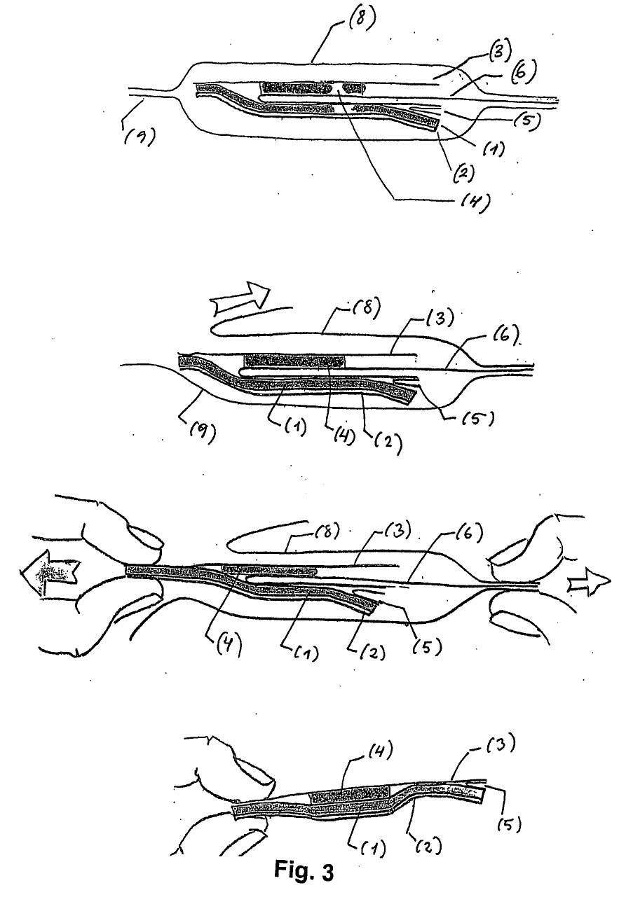

According to the above-mentioned preferred embodiment of the invention, the roller foil suitable has two layers. Where the roller foil has two layers, the second layer of the roller foil forms a tab member. Suitably the second layer is longer than the first layer of the roller foil and suitably it forms a tap portion extending beyond the periphery of the first adhesive zone of the adhesive layer. This embodiment of the invention is illustrated in FIG. 3.

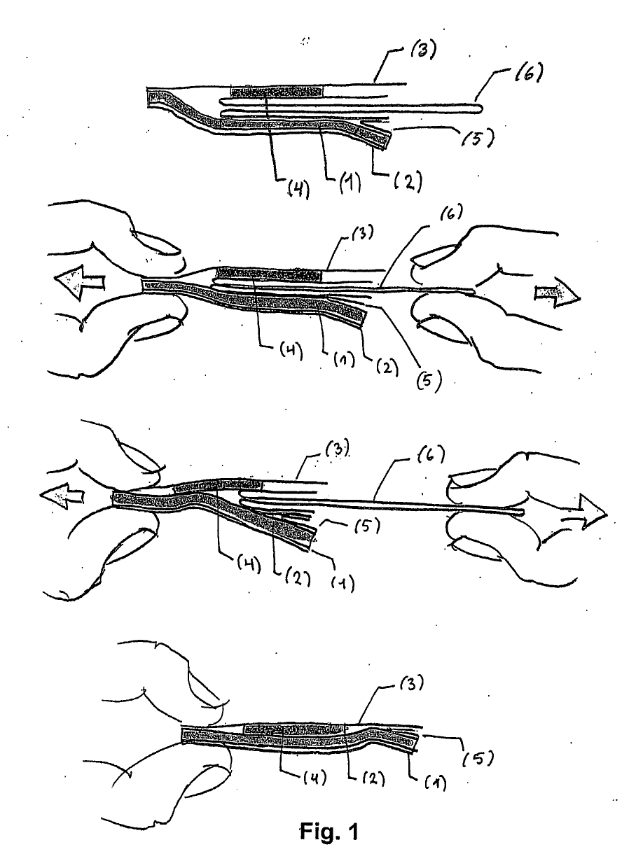

According to another embodiment of the invention, the roller foil has four layers, and the second and third layers form a tab member. In a preferred embodiment, the second and the third layer of the roller foil form is longer than the first layer of the roller foil, and form a tap portion. Suitably, the tap portion is extending beyond the periphery of the first adhesive portion of the adhesive layer. This embodiment of the invention is illustrated in FIG. 1.

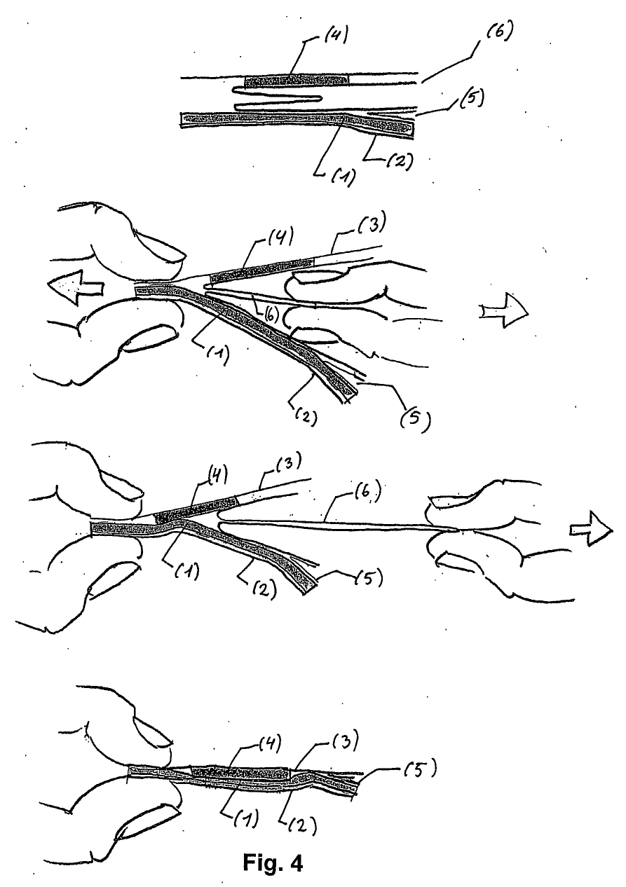

According to another embodiment of the invention, the first layer of the roller foil is longer than the second and third layer of the roller foil. The second and third layer may still be considered to form a tap member. This embodiment of the invention is illustrated in FIG. 4.

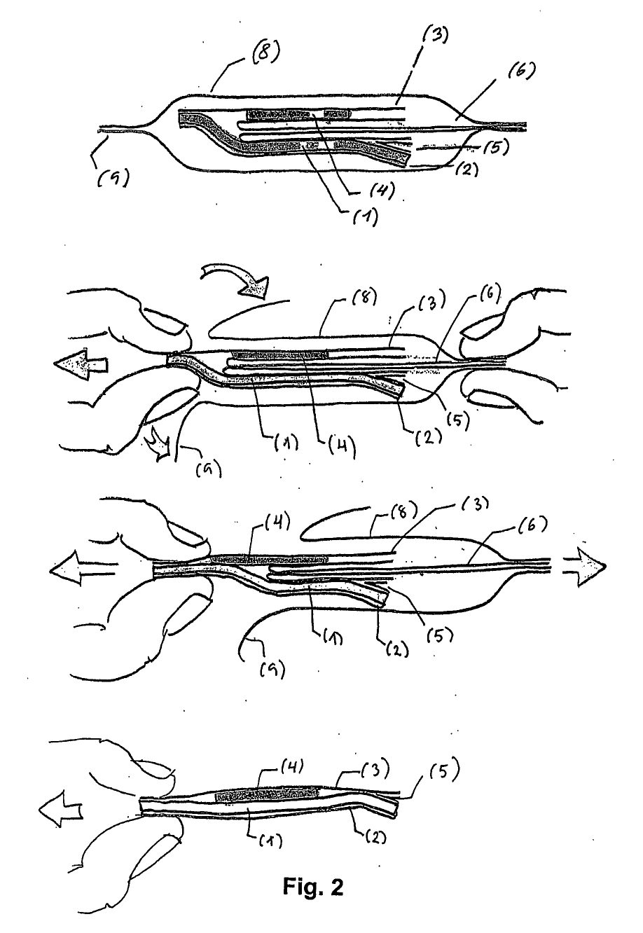

An alternative to the embodiment of the Invention where the roller foil has two layers, is an embodiment where the first layer and the second layer of a roller foil is positioned as described above and a third, preferably a third and fourth layer of another roller foil is positioned and aligned on top of the first and second layer of the roller foil, whereby the second and third layer of the roller foil form a tap portion. Suitably the second and third layers of the roller foils are longer than the first (and fourth) layer and are suitably extending beyond the periphery of the first adhesive portion of the adhesive layer. This embodiment of the invention is illustrated in FIG. 2.

The first layer of the roller foil may also be longer than the second and third layer of the roller foil. The second and third layer may still be considered to form a tap member.

The roller foil may also have three or more than four layers, but two or four layers are preferred. In particular four layers are preferred, when the element(s) carried by the second release layer has adhesive properties, because the roller foil may then be rolled of the element(s) instead of tearing it of.

The adhesive dressing of the invention may have different sizes, i.e. various surface areas.

As used herein, the expression that a surface area of a layer, e.g. a release layer “corresponds to or is corresponding to” an adhesive surface area” means that the surface are of the release layer is able to cover the adhesive surface in question completely.

The adhesive dressing according to the invention may have any suitable shape, such as rectangular, circular, ellipsoid, circular with tab portions etc. The shape of the adhesive layer is not particularly important. However, suitably the adhesive layer has an elongated form, e.g. a rectangular form or and ellipsoid form.

The area of the second release layer should be sufficiently large for at least the entire adhesive mid zone and optionally the second adhesive zone of the adhesive dressing to be placed safely and accurately thereon when the roller foil or third release layer is removed from the adhesive surface. Suitably, the second release layer has a shape and area corresponding to the adhesive layer or it is larger and has the same or a different shape.

The surface area and shape of the roller foil or third release layer is not particularly important as long as the surface area of the roller foil or third release layer is sufficiently large to accommodate at least the entire mid portion of the adhesive surface of the adhesive layer.

Suitably the roller foil is a layered item having an elongated shape, such as rectangular or ellipsoid.

Suitably, the portion of the first release layer, which is releasable attached to the adhesive layer, has an area and shape corresponding to the first adhesive zone of the adhesive layer to which it is releasable attached, or it may be a little larger and have the same shape or a different shape. Preferably, the portion of the first release layer, which is attached to the adhesive surface, has an area and shape corresponding to the first adhesive zone of the adhesive surface to which it is releasable attached.

The tab member formed by the folded first release layer should preferably be sufficiently large to be gripped by the fingertips of a human being.

Likewise, the second adhesive zone of the adhesive surface and the portion of the second release layer to which it is releasable attached, suitably has an area sufficiently large to be gripped by the fingers of a human.

Preferably the folding line(s) of the roller foil is perpendicular to an axis of symmetry through the foil from one end the roller foil to the opposite end of the roller foil. However, other alternatives exist.

According to the invention, the second release layer may carry one or more element(s) to be transferred to the adhesive surface of the adhesive layer, in particular the adhesive mid zone of the adhesive layer.

The element(s) may comprise one or more active substance(s), such a pharmaceutically active substance(s).

The above-mentioned element(s) may also be a hydrogel or any other absorbent material, or it may comprise a hydrogel, or any other absorbent material.

The element(s) may have an adhesive surface.

In one embodiment, the element(s) are covered by a barrier layer preventing the element(s) from coming into direct contact with the adhesive surface. The barrier layer may further eliminate or diminish migration of substances from the element(s) into the adhesive surface. The barrier layer may be a metal film, a polymer film (polyurethane of polyethylene film) or siliconized paper.

The second release layer may carry one single element releasable attached to the release layer. It is also possible that multiple elements of the same or of a different kind (e.g. different compositions) are releasable attached to the second release layer.

Multiple element(s) carried by the second release layer may be in the form of dots, circles, or may form stripes or any other pattern on the second release layer.

In one embodiment, the element(s) carried by the second release layer is contained in one or more cavities formed in the release layer.

The element(s) carried by the second release layer or layer may comprise one or more active substances, such as pharmaceutically active substances, vitamins, or any other active substance suitable for transdermal delivery or delivery to wounds.

The active substance may be emollients or e.g. retinoids for treating or preventing formation of psoriasis, eczema, callous, skin, corns or blisters, or medicaments, e.g. antiviral medicaments for treatment of herpes, acne and warts.

Suitably, the active substance is an active substance selected from a cytochine, a growth hormone or a polypetide growth factor such as TGF, FGF, PDGF, EGF, IGF-1, or IGF-2, a colony stimulating factor, a transforming growth factor, nerve stimulating growth factor, bacteriostatic or bactericidal compounds, e.g. iodine, iodopovidone complexes, chloramine, chlorhexidine, silver salts such as sulphadizine, silver nitrate, silver acetate, silver lactate, silver sulphate, silver sodium thiosulphate or silver chloride, metronidazol, sulpha drugs, and pencillins, anti viral medicaments for the treatment of herpes may for example comprise aciclovir or penciclovir, a medicament for the treatment of acne, such as azelain acid or isotretinoin, a medicament for treatment of warts, e.g a mitotic inhibitor, such as podophyllotoxin, salicylic acid-based medicaments for treating warts and/or clavus, tissue-healing enhancing agents, e.g. RGD tripeptides and the like, proteins, amino acids such as taurine, vitamins such as ascorbic acid, enzymes for cleansing of wounds, e.g. pepsin, trypsin and the like, proteinase inhibitors for use in e.g. surgical insertion of the dressing in cancer tissue and/or other therapeutic agents which optionally be used for topical application, pain relieving agents such as lidocaine or chinchocaine, emollients, retinoids or agents having a cooling effect.

The layered product of the invention allows for an accurate positioning of the element(s) carried by the second release layer of the adhesive layer. Suitably, the element(s) carried by the second release layer is placed in a position corresponding essentially to a final position in the centre or in the central part of the adhesive mid zone of the adhesive layer.

The active substances, including the pharmaceutically active substances, may be contained in a suitable vehicle when carried on the second release layer.

According to the invention, the elements carried by the second release layer may be applied to the adhesive surface simultaneously with the removal of the roller foil, and simultaneously with the removal of the adhesive article from an integrated package.

Thus, the invention also relates to a package having the layered product as above interposed between a top and a bottom layer, the top and bottom layer having a first peripheral end portion, a second peripheral end portion positioned distally to said first peripheral end portion, and peripheral side portions connecting the peripheral end portions, where said top and bottom layer are inseparably or releasable attached to each other in their first peripheral end portions and optionally in the peripheral side portions of the top and bottom layers.

The top and bottom layer of the package have a surface area, which is larger than the surface area of the layered product and by peripheral portions of the top and bottom layer is meant portions extending beyond the adhesive article interposed between the top and bottom layer.

In a preferred embodiment, the tab portion formed of the second layer of the roller foil, or a tap portion formed of the second and third layer of the roller foil is attached to the top layer, the bottom layer, or both the top and bottom layer, suitably in the first peripheral end portion of the top and bottom layer.

Suitably, the peripheral side portions of the top and bottom layer are releasable or inseparably attached to each other.

In a preferred embodiment the second peripheral end portions of the top and bottom layer are releasable attached to each other. In this case a sterile sealed package may be provided. According to this embodiment, the second peripheral end portions of the top and bottom layer are suitably releasable attached to each other at a distance from the most proximate end edges of the top and bottom layer, in order to provide portions of the top and bottom layer which may be gripped for pulling the top and bottom layer apart and thereby opening the package.

The invention also relates to a method for laminating the second release layer onto the adhesive layer of the adhesive article contained within the package comprising the steps of

a) Optionally opening the package;

b) Gripping the portion where the second adhesive zone of the adhesive layer is releasable attached to the second release layer, gripping the end portion of the package to which the extending portion of the roller foil is attached, and pulling the article out of the package, simultaneously removing the roller foil and laminating the adhesive mid zone of the adhesive layer to the second release layer; and optionally

c) Separating the second release layer from the adhesive mid zone and the second adhesive zone of the adhesive layer,

d) Applying the mid adhesive zone and the second adhesive zone of the adhesive layer to the anatomical surface of a living being; and

e) Gripping the tab member of the first release layer and removing the first release layer from the first adhesive zone of the adhesive layer.

According to another embodiment of the invention, the second release surface is laminated to the adhesive surface by

a) Optionally opening the package;

b) Pulling the layered product out of the package,

c) Removing the roller foil or third release layer from the adhesive layer, and laminating at least the adhesive mid zone of the adhesive layer to the second release layer; and optionally

d) Separating the second release layer from the adhesive mid zone and the second adhesive end zone of the adhesive layer;

e) Applying the adhesive mid zone and the second adhesive end zone of the adhesive surface to the anatomical surface of a living being; and

f) Gripping the tab member of the first release layer and removing the first release layer from the first adhesive zone of the adhesive layer.

The adhesive dressing according to the present invention suitably comprises a pressure sensitive adhesive formulated from materials suitable for use on human skin and a flexible cover layer. Suitable pressure sensitive adhesive compositions are homogenous blends of one or more polyisobutylenes or mixtures of one or more polyisobutylenes and an elastomer such as butyl rubber, butadiene and styrene block copolymers (S-B-S), polyisoprene and styrene block copolymers (S-I-S), Styrene polyisobutylene block copolymers (SIBS) or any other known adhesive compositions useful for adhesive dressings.

The adhesive composition may contain one or more moisture absorbing or moisture transmitting, water-soluble and/or water swellable agents. Such agents include hydrocolloids and are known in the art.

The adhesive composition may also contain other ingredients such as plastizicers, tackyfiers, antioxidants etc.

Suitable cover layers include polymeric films, such as films made from polyethylene, polyurethane, polyether polyamide block copolymers etc, as well as woven and non-woven fabric layers.

The release layers may be made from paper or cardboard material, which may optionally be coated with one or more coatings providing the desired adhering properties, such as releasability. Alternatively the release layers may be a film made from a metal, such aluminum, or from a plastic material, for example a polyester, such as polyethyleneterephthalate (PETP).

The adhering properties (or releasability) of the release layer may vary in dependence of the choice of material for the coating layer. Suitable material groups may e.g. comprise silicone, metals, and Teflon™. It has been found that silicone materials are generally well suited for the coating, as various available silicone materials provide a useful variety of adhering properties. It is a matter of routine experimentation to identify the specific material, which is best, suited for a particular embodiment or purpose.

The second release layer is suitably siliconized paper.

The first release layer may be siliconized paper or a plastic foil.

The top and bottom layers are suitably made of paper or any other material suitable for the purpose.

The roller foil is suitably a plastic foil, e.g. made from a polyester, such as PETP, or a metal foil, such as aluminum.

The required releasability of the foil or release layers may be controlled by appropriate selection of a material or of the coating thereof, such as for example a silicone coating. In order to facilitate rolling of the foil and thus transfer of the mid zone of the adhesive layer to the second release layer, the foil should be soft and flexible. The roller foil is suitably a non-elastic plastic foil.

The thickness of the adhesive is in the range of 0.1-2.5 mm, preferably 0.2-0.8 mm and the vapor permeability of the dressing sheet preferably being 300-800 g/m²/24 h.

The release layers, including those made from an optionally silicone coated paper, cardboard or plastics material may typically have a thickness of 20-150 μm.

The outer periphery of the adhesive dressing may be beveled in order to reduce the risk of rolling-up the dressing, which would reduce wear-time.

In one embodiment, the invention relates to a layered product comprising:

(I) an adhesive dressing comprising an adhesive layer having a cover layer inseparably attached to one surface of said adhesive layer, the other surface of said adhesive dressing consisting of a first adhesive zone and a second adhesive zone,

(II) a bottom release layer having a portion thereof attached to the first adhesive zone of the adhesive dressing and having a tap portion,

(III) at least one roller foil having two or more layers folded on top of each other, and having a first layer with a first outer surface releasable attached to at least the second adhesive zone of the adhesive dressing and a second layer forming a tab portion or forming a part of a tab portion, and wherein the folded edge between the first and the second layer of the roller foil(s) is positioned at the boundary between the first and second zone of the adhesive surface of the adhesive dressing, and wherein

(IV) one or more element(s) is interposed between said bottom release layer and the roller foil(s), said one or more element(s) being releasable attached to second outer surface of the roller foil(s).

According to one preferred embodiment, the roller foil has two layers and the end of the second layer form the tap portion. According to a suitable embodiment of the invention, the second layer of the roller foil is longer than the first layer, and in one particular embodiment the second layer of the roller foil is extending beyond the periphery of the second adhesive zone of the adhesive layer.

According to another embodiment of the invention, the roller foil has four layers, and the second and third layer form the tap portion.

According to still another embodiment of the invention, the layered product comprises two roller foils, each with two layers, the second and third layer thereof forming the tab portion.

According to these embodiments of the invention, the second and third layer of the roller foil(s) is suitably longer than the first layer of the roller foil and suitably the second and third layer of the roller foil is extending beyond the periphery of the second adhesive zone of the adhesive layer.

Preferably the folded edge between the third and fourth layer of the roller foil(s) is aligned with the folded edge between the first and second layer of the roller foil(s).

The first layer of the roller foil may also be longer than the second and third layer of the roller foil. The second and third layer may still form a tap portion.

The roller foil may also have three or more than four layers, but two or four layers are preferred. Four layers are preferred, in particular when the one or more element(s) have adhesive properties, because the roller foil(s) may then be rolled of the element(s) instead of tearing it off.

The area of the bottom release layer should be larger than the area needed to cover at least the one or more elements interposed between this layer and the roller foil(s). Suitably, the bottom release layer has a shape and area corresponding to the adhesive dressing or it is larger or smaller and has the same or a different shape.

Suitably, the portion of the bottom release layer, which is releasable attached to the adhesive layer, has an area and shape corresponding to the first adhesive zone of the adhesive dressing to which it is releasable attached, or it may be a little larger and have the same shape or a different shape. Preferably, the portion of the bottom release layer, which is attached to the adhesive surface, has an area and shape corresponding to the first adhesive zone of the adhesive dressing to which it is releasable attached.

According to the invention, the one or more element(s) to be transferred to the skin and to the adhesive surface of the adhesive dressing is placed in a position between the roller foil(s) and the bottom release layer corresponding to a final position in the central part of the adhesive dressing.

In one embodiment, one single element may be releasable attached to the roller foil(s). It is also possible that multiple elements of the same or of a different kind (e.g. different compositions) are releasable attached to the roller foil(s).

The layered product of the invention allows for an accurate positioning of the element(s) under the adhesive dressing. Suitably, the element(s) is placed in a position corresponding essentially to a final position in the centre or in the central part of the adhesive dressing.

According to the invention, the element(s) may be applied to the adhesive surface immediately after placing the one or more elements on the skin of a living being. Application of the adhesive dressing to the one or more elements may be carried out simultaneously with the removal of the roller foil, and in some case simultaneously with the removal of packaging material.

The invention also relates to a package having the layered product as above interposed between a top and a bottom layer, the top and bottom layer having a first peripheral end portion, a second peripheral end portion positioned distally to said first peripheral end portion, and peripheral side portions connecting the peripheral end portions, where said top and bottom layer are inseparably or releasable attached to each other in their first peripheral end portions and optionally in the peripheral side portions of the top and bottom layers.

According to one particular embodiment of the invention the bottom release layer form the bottom layer of the package. According to this embodiment of the invention the layered product as above is interposed between a top layer and the bottom release layer, the top layer and the bottom release layer having a first peripheral end portion, a second peripheral end portion positioned distally to said first peripheral end portion, and peripheral side portions connecting the peripheral end portions, where said top layer and said release layer are inseparably or releasable attached to each other in their first peripheral end portions and optionally in the peripheral side portions of the top and bottom layers.

According to this embodiment, the top and bottom layer (e.g. the first adhesive layer) of the package have a surface area, which is larger than the surface area of the adhesive dressing and by peripheral portions of the top and bottom layer (e.g. the first adhesive layer) is meant portions extending beyond the adhesive dressing interposed between the top and bottom layer.

In a preferred embodiment, the tab portion formed of the second layer of the roller foil, or a tap portion formed of the second and third layer of the roller foil is attached to the top layer, the bottom layer (or bottom release layer), or both the top and bottom layer (bottom release layer), suitably in the first peripheral end portion of the top and bottom layer (bottom release layer).

Suitably, the peripheral side portions of the top and bottom layer (bottom release layer) are releasable or inseparably attached to each other. Preferably the side portions are separable attached to each other.

Methods for attaching the sides of a package, whether it is by releasable attachment or inseparable attachment are well described, and include welding, adhesion, cohesion, etc.

In a preferred embodiment the second peripheral end portions of the top and bottom layer are releasable attached to each other. In this case a sterile sealed package may be provided. According to this embodiment, the second peripheral end portions of the top and bottom layer (bottom release layer) are suitably releasable attached to each other at a distance from the most proximate end edges of the top and bottom layer, in order to provide portions of the top and bottom layer which may be gripped for pulling the top and bottom layer apart and thereby opening the package.

The invention also relates to method for the application of the layered product as above to the skin of a living being, comprising the steps of:

a) Removing the bottom release layer from said one or more element(s), and optionally form the first adhesive zone of the adhesive dressing,

b) Placing the said one or more elements and optionally the second adhesive zone of the adhesive dressing on the skin,

c) Gripping the tab portion of the roller foil(s) and removing the roller foil(s) from the one or more element(s) and the second adhesive zone of the adhesive dressing, and pressing the adhesive dressing against the skin.

The invention also relates to a method for the application of the adhesive dressing comprised in the package as above to the skin of a living being comprising the steps of:

a) Optionally opening the package;

b) Removing the bottom release layer from said one or more element(s), and optionally form the first adhesive zone of the adhesive dressing,

c) Placing the said one or more elements and optionally the second adhesive zone of the adhesive dressing on the skin,

d) Gripping the tab portion of the roller foil(s) or gripping the end portion of the top and/or bottom layer at the end where the tap portion is attached, and removing the roller foil from the one or more element(s) and the first adhesive zone of the adhesive dressing, and pressing the adhesive dressing against the skin.

The bottom release layer is suitably siliconized paper or it may be a plastic foil.

The top and bottom layers are suitably made of paper or any other material suitable for the purpose.

The required releasability of the foil or release layers may be controlled by appropriate selection of a material or of the coating thereof, such as for example a silicone coating. In order to facilitate rolling of the foil, the foil should be soft and flexible. The roller foil is suitably a non-elastic plastic foil.

Suitably, the strength of the bond between the one or more elements to the roller foil(s) is stronger than the bond between the bottom release layer and the one ore more element(s), and suitable the bond between said one or more element(s) and the skin is stronger than the bond to the roller foil.

### DETAILED DESCRIPTION OF THE INVENTION

FIG. 1 illustrates a layered product of the invention comprising an adhesive dressing having an adhesive layer (1) and a cover layer (2), a first release layer (5) releasable attached to a first adhesive zone of the adhesive dressing and folded upon itself to form a tap member having the folded edge positioned at the boundary between the adhesive mid zone of the adhesive dressing and the first adhesive zone of the adhesive dressing, a second release layer (3) carrying an element (4) to be transferred from the second release layer to the adhesive surface of the adhesive dressing, and a roller foil (6) with four layers, where the first layer is releasable attached to the adhesive mid zone of the adhesive dressing, and the second and third layer forming a tab member which is longer than the first layer of the roller foil and is extending beyond the peripheral end edge of the first adhesive zone of the adhesive layer.

The figure illustrates how the roller foil is removed and the second release layer laminated onto the adhesive layer.

FIG. 2 illustrates a layered product as in FIG. 1 (with the exception that the four layers of the roller foil consists of two roller foils each having two layers) placed within a package having a top layer (8) and a bottom layer (9).

The figure also illustrates the simultaneous removal of the adhesive article from the package, removal of the roller foil(s) and lamination of the second release layer onto the adhesive layer.

FIG. 3 illustrates the same as FIG. 2 with the exception that the layered product comprises one roller foil with two layers.

FIG. 4 illustrates a layered product of the invention where the roller foil (6) has four layers, where the second and third layer is shorter than the first (and fourth) layer.

The figure illustrates how the roller foil is removed and the second release layer laminated onto the adhesive layer.

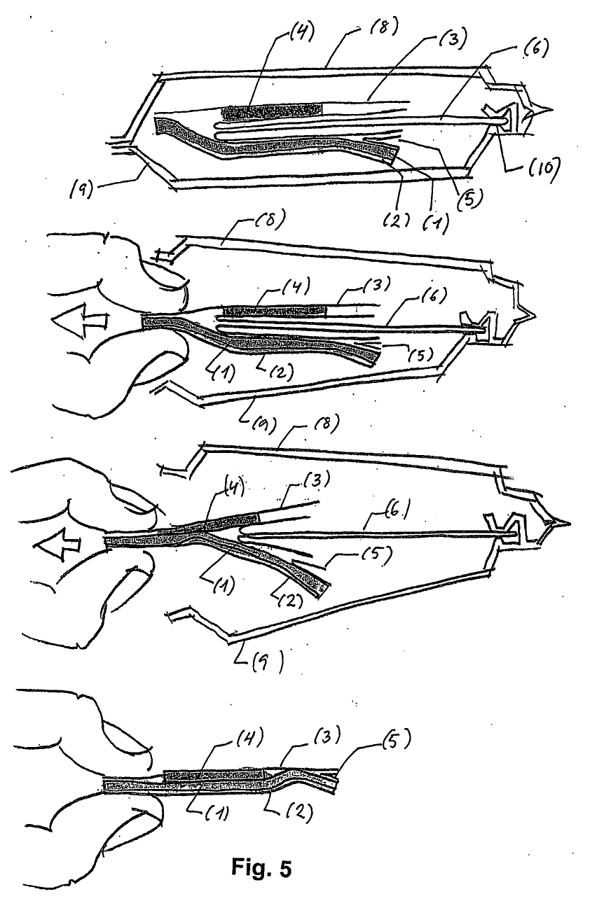

FIG. 5 illustrates a package comprising the layered product of FIG. 1 interposed between a top layer (8) and a bottom layer (9) where the second and third layer of the roller foil (6) is welded (10) to the bottom layer at the peripheral end portion of these layers.

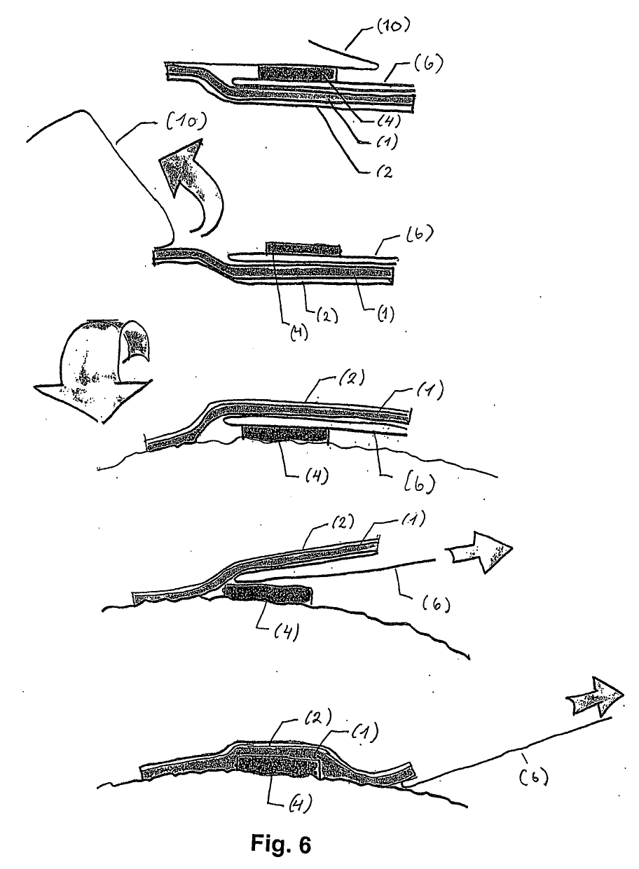

In FIGS. 6-8 is illustrated an embodiment of the invention wherein the dressing is combined with the element(s) during application to the skin. FIG. 6 illustrates a layered product of the invention comprising an adhesive dressing having an adhesive layer (1) and a cover layer (2), an element (4) interposed between a bottom release layer (10) releasable attached to a first adhesive zone of the adhesive dressing and a roller foil (6) with two layers having the folded edge positioned at the boundary between the second adhesive zone of the adhesive dressing and the first adhesive zone of the adhesive dressing and a tab portion formed from the second layer of the roller foil.

The figure illustrates how the bottom release layer (10) is removed, the element (4) applied to the skin and the roller foil (6) is removed and the adhesive layer (1) is laminated onto the element (4).

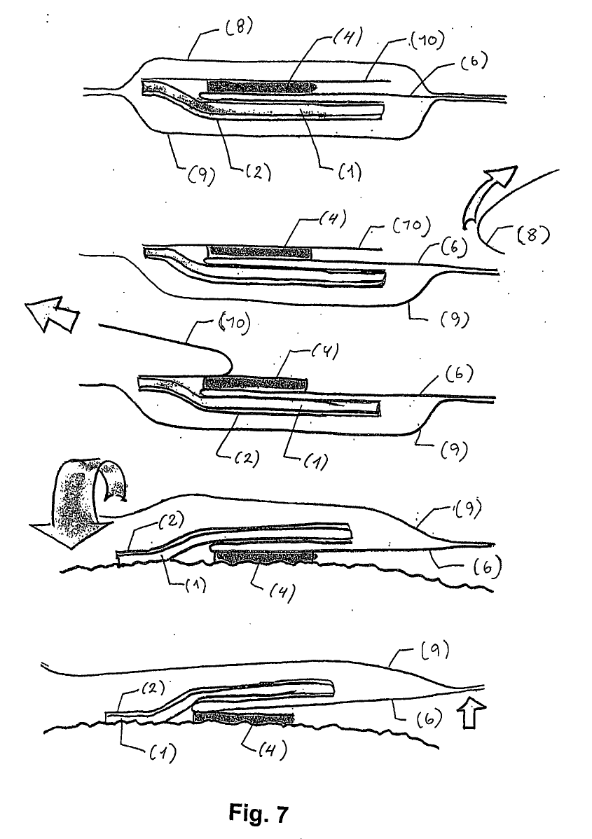

FIG. 7 illustrates a layered product as in FIG. 6 (with the exception that the four layers of the roller foil consists of two roller foils each having two layers) placed within a package having a top layer (8) and a bottom layer (9).

The figure also illustrates the opening of the package, removal of the bottom release layer (10) from the element (4) followed by application of the element (4) and the first adhesive zone of the adhesive dressing to the skin, and thereafter removal of the roller foil(s) (6) and the rest of the package from the adhesive dressing and application of the second adhesive zone of the adhesive dressing onto the element (4) and the skin.

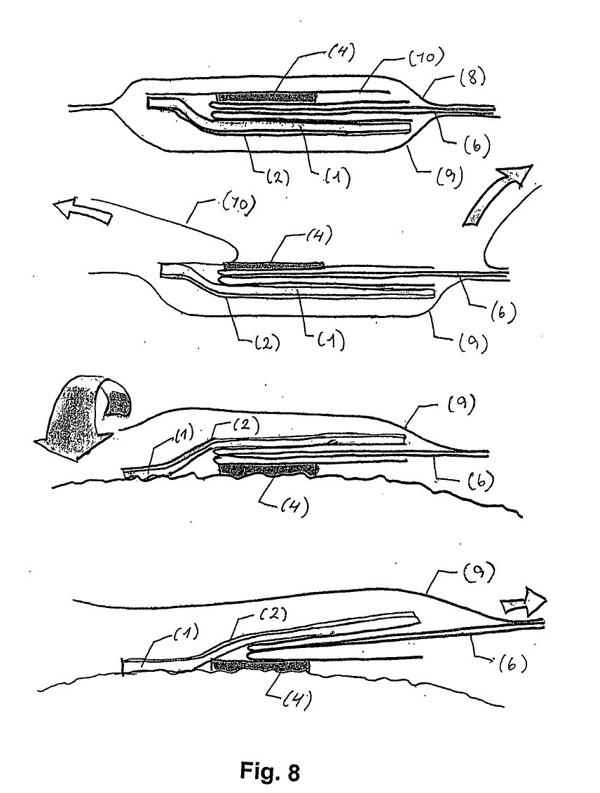

FIG. 8 illustrates the same as FIG. 7 with the exception that the layered product comprises one roller foil with two layers.

1: A layered product comprising:
(I) an adhesive dressing comprising an adhesive layer having a cover layer inseparably attached to one surface of said adhesive layer, the other surface of said adhesive layer consisting of a first adhesive zone, a second adhesive zone positioned distally to the first adhesive zone, and an adhesive mid zone positioned between the first and second adhesive zones,
(II) a first release layer releasable attached to the first adhesive zone of the adhesive dressing, said first release layer being folded upon itself to form a tap member and having the folded edge positioned at the boundary between the adhesive mid zone of and the first adhesive zone of the adhesive dressing,
(III) a second release layer having a surface area corresponding to at least the surface area of the mid zone and optionally the first and/or second zones of the adhesive dressing, said second release layer optionally carrying one or more element(s) releasable attached to the second release layer on a first surface of the second release layer
characterized in that
a roller foil or third release layer having a tab member is releasable attached to at least the adhesive mid zone of the adhesive dressing, and
in that the adhesive dressing and the second release layer are connected to each other by means allowing positioning of said first surface of the second release layer on the adhesive mid zone and optionally the second adhesive zone of the adhesive dressing by folding.

2: The layered product according to claim 1 wherein the adhesive dressing and the second release layer lies in extension of each other and are connected to each other by one of their edges, the connection between the edges providing a folding line or folding centre allowing positioning of said first surface of the second release layer on the adhesive mid zone and optionally the second adhesive zone of the adhesive dressing.

3: The layered product according to claim 2 wherein the adhesive dressing and the second release layer lies in extension of each other and is carried on a support layer fixing the adhesive dressing and the second release layer at a distance from each other, where said support layer provides a folding line or center allowing positioning of said first surface of the second release layer on the adhesive mid zone and optionally the second adhesive zone of the adhesive dressing.

4: The layered product according to claim 1 wherein the roller foil or third release layer is a single layer releasable attached to at least the entire adhesive mid portion of said adhesive dressing and optionally to any adhesive element(s) carried by the second release layer.

5: The layered product according to claim 1 wherein the tap member of the roller foil or third release layer is an end portion of the roller foil or third release layer.

6: The layered product according to claim 1 wherein the tap member of the roller foil or third release layer is a folded portion of the roller foil or third release layer.

7: The layered product according to claim 6 wherein a first and a third folding line of said folded portion is situated on top of and aligned with the folding line allowing positioning of the second release layer on the adhesive mid zone of the adhesive dressing by folding.

8: The layered product according to claim 1 wherein the second adhesive zone of the adhesive layer is releasable attached to an end portion of said second release layer.

9: The layered product according to claim 1 wherein the roller foil or third release layer is interposed between the second release layer and at least the adhesive mid zone of the adhesive dressing.

10: The layered product according to claim 9 wherein roller foil or third release layer is a single layer interposed between the second release layer and at least the adhesive mid zone of the adhesive dressing.

11: The layered product according to claim 9 wherein the roller foil or third release layer has two or more layers interposed between the second release layer and at least the adhesive mid zone of the adhesive dressing.

12: The layered product according to claim 8 characterized in that it comprises
a roller foil having two or more layers folded on top of each other interposed between the second release layer and at least the adhesive mid zone of the adhesive dressing, where the outer surface of the first layer of said folded roller foil is releasable attached to the mid zone of the adhesive dressing and the folded edge between said first layer of the roller foil and the second layer of the roller foil is positioned at the boundary between the second adhesive zone and the adhesive mid zone of the adhesive layer.

13: The layered product according to claim 12 wherein the roller foil has two layers and the second layer form the tap member.

14: The layered product according to claim 13 wherein the second layer of the of the roller foil is longer than the first layer.

15: The layered product according to claim 14 wherein the second layer of the of the roller foil is extending beyond the periphery of said first adhesive zone of the adhesive layer

16: The layered product according to claim 12 wherein the roller foil has four layers, and the second and third layer form the tap member.

17: The layered product according to claim 13, wherein an additional roller foil having two layers providing a third and fourth layer is interposed between the second release layer and at least the adhesive mid zone of the adhesive dressing, and the second and third layer thereof form the tab member.

18: The layered product according to claim 16 wherein the second and third layer of the roller foil(s) is longer than the first layer of the roller foil.

19: The layered product according to claim 18 wherein the second and third layer of the roller foil is extending beyond the periphery of said first adhesive zone of the adhesive layer.

20: The layered product according to claim 16 wherein the second and third layer of the roller foil(s) is shorter than the first layer of the roller foil.

21: The layered product according to claim 1 wherein the second release layer carries one or more element(s) to be transferred onto the adhesive surface of the adhesive layer.

22: The layered product according to claim 20 wherein the element(s) comprises one or more active substance(s), such a pharmaceutically active substance(s).

23: A layered product according to claim 21 where the element(s) comprises a hydrogel or a water absorbent material.

24: A layered product according to claim 21 wherein the element(s) has an adhesive surface.

25: A layered product according to claim 21 where the element(s) is covered by a barrier layer.

26: A package having the layered product according to claim 1 interposed between a top and a bottom layer, the top and bottom layer having first peripheral end portions, second peripheral end portions positioned distally to said first end portions, peripheral side portions connecting said peripheral end portions, where said top and bottom layer are inseparably or releasable attached to each other at least at the first peripheral end portions of the top and bottom layers.

27: A package having a layered product according to claim 12 interposed between a top and a bottom layer, the top and bottom layer having first peripheral end portions, second peripheral end portions positioned distally to said first peripheral end portions, and peripheral side portions connecting said peripheral end portions, where said the top and bottom layer is inseparably or releasable attached to each other at least at said first peripheral end portions of the top and bottom layers.

28: The package according to claim 27 wherein a tap portion formed of the second layer of the roller foil, or a tap portion formed of the second and third layer of the roller foil is attached to the top layer, the bottom layer, or to both the top and bottom layer at the first peripheral end portion(s) of the top and bottom layer.

29: The package according to claim 26 wherein the peripheral side portions of the top and bottom layer are separable or inseparably attached to each other.

30: The package according to claim 26 wherein the second peripheral end portions of the top and bottom layer are releasable sealed to each other, suitably at a distance from the second end edges of the top and bottom layer, in order to provide portions of the top and bottom layer which may be gripped for pulling the top and bottom layer apart and thereby opening the package.

31: A method for the removal of the roller foil(s) from the layered product contained within a package according to claim 28 comprising the steps of
a) Optionally opening the package;
b) Gripping the portion where the second adhesive zone of the adhesive layer is releasable attached to the second release layer, gripping the end portion of the package to which the extending portion of the roller foil is attached, and pulling the article out of the package, simultaneously removing the roller foil and laminating the adhesive mid zone of the adhesive layer to the second release layer; and optionally
c) Separating the second release layer from the adhesive mid zone and the second adhesive zone of the adhesive layer,
d) Applying the mid adhesive zone and the second adhesive zone of the adhesive layer to the anatomical surface of a living being, and
e) Gripping the tab member of the first release layer and removing the first release layer from the first adhesive zone of the adhesive layer.

32: A method for removal of the third release layer or roller foil from the layered product contained within a package according to claim 26 comprising the steps of
a) Optionally opening the package;
b) Pulling the layered product out of the package;
c) Removing the roller foil or third release layer from the adhesive layer, and
laminating at least the adhesive mid zone of the adhesive layer to the second release layer; and optionally
d) Separating the second release layer from the adhesive mid zone and the second adhesive end zone of the adhesive layer;
e) Applying the adhesive mid zone and the second adhesive end zone of the adhesive surface to the anatomical surface of a living being; and
f) Gripping the tab member of the first release layer and removing the first release layer from the first adhesive zone of the adhesive layer.

33: A layered product comprising:
(I) an adhesive dressing comprising an adhesive layer having a cover layer inseparably attached to one surface of said adhesive layer, the other surface of said adhesive dressing consisting of a first adhesive zone and a second adhesive zone,
(II) a bottom release layer having a tap portion,
(III) one or more roller foil(s) each having two or more layers folded on top of each other, and having a first layer with a first outer surface releasable attached to at least the second adhesive zone of the adhesive dressing, and a second layer forming a tab portion or forming part of a tab portion, wherein
one or more element(s) is interposed between said bottom release layer and the roller foil, said one or more element(s) being releasable attached to the second outer surface of the roller foil(s).

34: The layered product according to claim 33 wherein the first adhesive zone of the adhesive dressing is releasable attached to the bottom release layer.

35: The layered product according to claim 34 wherein the folded edge between the first and the second layer of the roller foil(s) is positioned at the boundary between the first and second zone of the adhesive surface of the adhesive dressing.

36: The layered product according to claim 33 comprising one roller foil with two layers and the second layer form a tap portion.

37: The layered product according to claim 33 wherein the second layer of the roller foil is longer than the first layer.

38: The layered product according to claim 36 wherein the second layer of the roller foil is extending beyond the periphery of the second adhesive zone of the adhesive layer

39: The layered product according to claim 33 comprising one roller foil with four layers, and the second and third layer form the tap portion.

40: The layered product according to claim 33, comprising two roller foils each having two layers and the second and third layer thereof form the tab portion.

41: The layered product according to claim 39 wherein the second and third layer of the roller foil(s) is longer than the first layer of the roller foil.

42: The layered product according to claim 40 wherein the second and third layer of the roller foil is extending beyond the periphery of the second adhesive zone of the adhesive layer.

43: The layered product according to claim 39 wherein the second and third layer of the roller foil(s) is shorter than the first layer of the roller foil.

44: The layered product according to claim 33 wherein the one or more element(s) interposed between the roller foil and the bottom release layer have an adhesive surface.

45: The layered product according to claim 33 wherein the element(s) comprises one or more active substance(s), such a pharmaceutically active substance(s).

46: A layered product according to claim 33 where the element(s) comprises a hydrogel or a water absorbent material.

47: A layered product according to claim 33 where a barrier layer is interposed between the roller foil(s) and the one or more elements.

48: A package having the layered product according to claim 33 interposed between a top and a bottom layer, the top and bottom layer having first peripheral end portions, second peripheral end portions positioned distally to said first end portions, peripheral side portions connecting said peripheral end portions, where said top and bottom layer are inseparably or releasable attached to each other at least at the first peripheral end portions of the top and bottom layers.

49: A package according to claim 48 wherein the bottom release layer is the bottom layer.

50: The package according to claim 48 wherein the tap portion formed of the second layer of the roller foil, or a tap portion formed of the second and third layer of the roller foil is attached to the top layer, the bottom layer or to both the top and bottom layer at the first peripheral end portion(s) of the top layer and bottom layer

51: The package according to claim 48 wherein the peripheral side portions of the top and bottom layer are separable or inseparably attached to each other.

52: The package according to claim 48 wherein the second peripheral end portions of the top and bottom layer are releasable attached to each other, suitably at a distance from the second end edges of the top and bottom layer, in order to provide portions of the top and bottom layer which may be gripped for pulling the top and bottom layer apart and thereby opening the package.

53: A method for the application of the layered product according to claim 33 to the skin of a living being, comprising the steps of:
a) Removing the bottom release layer from said one or more element(s), and optionally form the first adhesive zone of the adhesive dressing,
b) Placing the said one or more elements and optionally the second adhesive zone of the adhesive dressing on the skin,
c) Gripping the tab portion of the roller foil(s) and removing the roller foil(s) from the one or more element(s) and the second adhesive zone of the adhesive dressing, and pressing the adhesive dressing against the skin.

54: A method for the application of the adhesive dressing comprised in the package according to claim 48 to the skin of a living being comprising the steps of:
a) Optionally opening the package;
b) Removing the bottom release layer or the bottom layer from said one or more element(s), and optionally form the first adhesive zone of the adhesive dressing,
c) Placing the said one or more elements and optionally the second adhesive zone of the adhesive dressing on the skin,
d) Gripping the tab portion of the roller foil(s), or gripping the end portion of the top and/or bottom layer at the end where the tap portion is attached, and removing the roller foil from the one or more element(s) and from the first adhesive zone of the adhesive dressing, and pressing the adhesive dressing against the skin.
